(19) United States
(12) Patent Application Publication
(10) Pub. No.: US 2021/0007985 A1
(43) Pub. Date: Jan. 14, 2021

(54) LIPOSOMAL ANTI-INFECTIVE FORMULATIONS TO INHIBIT NON-TUBERCULOUS MYCOBACTERIA (NTM) MICROAGGREGATE FORMATION AND ESTABLISHMENT OF NTM BIOFILM

(71) Applicants: ARADIGM CORPORATION, Hayward, CA (US); OREGON STATE UNIVERESITY, Corvallis, OR (US)

(72) Inventors: Igor GONDA, San Francisco, CA (US); James BLANCHARD, El Granada, CA (US); David C. CIPOLLA, San Ramon, CA (US); Luiz Eduardo Moreira BERMUDEZ, Corvallis, OR (US)

(73) Assignees: ARADIGM CORPORATION, Hayward, CA (US); OREGON STATE UNIVERSITY, Corvallis, OR (US)

(21) Appl. No.: 16/603,045

(22) PCT Filed: Mar. 12, 2018
(86) PCT No.: PCT/US2018/022031

§ 371 Date: Oct. 4, 2019

Related U.S. Application Data
(60) Provisional application No. 62/481,984, filed on Apr. 5, 2017.

(51) Int. Cl. A61K 9/127; A61K 31/496

(52) U.S. Cl. CPC A61K 9/127; A61K 9/0073; A61K 31/496

(57) ABSTRACT

Methods of treatment to prevent NTM microaggregate formation using formulations of liposomal ciprofloxacin. Specific liposome formulations and delivery of such for treatment of respiratory tract infections and other medical conditions, and devices and formulations used in connection with such are described.

### GOVERNMENT RIGHTS

This invention was made with government support under R43-AI106188 awarded by National Institutes of Health. The government has certain rights in the invention.

### FIELD OF THE INVENTION

The present invention relates to pharmaceutical compositions of liposomal anti-infectives, particularly liposomal quinolones and fluoroquinolones and liposomal aminoglycosides, for inhalation to prevent the initiation or formation of microaggregates of a variety of microorganisms or intracellular pathogens, particularly non tuberculous mycobacteria (NTM), and inhibiting the establishment of biofilms of NTM.

### BACKGROUND OF THE INVENTION

Respiratory tract infections are caused by a variety of microorganisms. Infections which are persistent have a myriad of consequences for the community including increased treatment burden and cost, and for the patient in terms of more aggressive treatment paradigms and potential for serious illness or even death. The formation of biofilms of pathogen bacteria in particular leads often to more pervasive infections that are difficult to treat. Examples of bacteria that form harmful biofilms are Pseudomonas aeruginosa and NTM. It would be beneficial if improved treatments were available to provide prophylactic treatment to prevent susceptible patients from acquiring new or different respiratory tract infections and prevent the formation of biofilm in the respiratory tract. It would also be beneficial for the new treatment to increase the rate or effectiveness of eradication for patients already infected with the microorganisms. The initial step in the formation of bacterial biofilms, such as those formed by NTM, are microaggregates of NTM. Patients who have previously had an episode of NTM infection may benefit by taking this therapy to prevent recolonization with NTM through inhibition of NTM microaggregate formation. In addition, people who had previously been treated for other infections of the respiratory tract may benefit from this therapy to prevent microaggregate formation of NTM and reduce the likelihood for NTM biofilm or colonization to occur or reoccur. NTM and Pseudomonas aeruginosa infections occur sometime simultaneously in the same patient. The patients who are treated with an inhaled antibiotic to control or eradicate respiratory infections with Pseudomonas aeruginosa but are as yet uninfected with NTM, may also benefit through the prophylactic action of the same judiciously selected inhaled antibiotic to prevent the formation of NTM microaggregates that could lead to NTM biofilms.

Pulmonary infections with non-tuberculosis mycobacteria (NTM) are notoriously difficult to treat. They exist in the lungs in various forms, including within macrophages and in biofilms. These locations are particularly difficult to access with antibiotics. Furthermore, the NTM may be either in a dormant (termed sessile), or a replicating phase, and an effective antibiotic treatment would target both phases. It was shown previously that formulations of ciprofloxacin and liposomal ciprofloxacin were efficacious against M. avium and M. abscessus in biofilm and macrophage assays (Blanchard et al., 2014; Bermudez et al., 2016) and in mouse lung infection models of M. avium (Bermudez et al., 2015) and M. abscessus (Blanchard et al., 2015).

We have found, surprisingly, that certain compositions of antibiotics including ciprofloxacin encapsulated in liposomes are effective in their antibacterial activity against the formation of microaggregates of NTM, the first step in NTM biofilm formation, and thus may provide both prophylactic as well as treatment benefits.

Lung infection from Mycobacterium avium subspecies hominissuis (hereafter referred as M. avium) and Mycobacterium abscessus (hereafter referred to as M. abscessus) is a significant health care issue and there are major limitations with current therapies. The incidence of pulmonary infections by NTM is increasing (Adjemian et al., 2012; Henkle et al., 2015; Prevots et al, 2010), specifically with M. avium and M. abscessus (Inderlied et al, 1993). About 80% of NTM in US is associated with M. avium (Adjemian et al., 2012; Prevots et al, 2010). M. abscessus, which is amongst the most virulent types, ranks second in incidence (Ballarino et al., 2009; Prevots et al, 2010). Diseases caused by both mycobacteria are common in patients with chronic lung conditions, e.g., emphysema, cystic fibrosis, and bronchiectasis (Yeager and Raleigh, 1973). They may also give rise to severe respiratory diseases, e.g., bronchiectasis (Fowler et al, 2006).

The infections may be from environmental sources and cause progressive compromising of the lung. Current therapy often fails on efficacy or is associated with significant side-effects. M. avium infection is usually treated with systemic therapy with a macrolide (clarithromycin) or an azalide (azithromycin) in combination with ethambutol and amikacin. In a recent Phase 2 clinical trial of liposomal amikacin for inhalation (ARIKAYCE™) in patients with treatment for refractory NTM infection, encouraging sputum conversion results were seen for M. avium but not for M. abscessus (Olivier et al., 2017; Winthrop et al, 2015). Oral or IV quinolones, such as ciprofloxacin and moxifloxacin, can be used in association with other compounds (Yeager and Raleigh, 1973), but higher intracellular or airway surface drug levels need to be achieved for maximal efficacy. Oral ciprofloxacin has clinical efficacy against M. avium only when administered in combination with a macrolide or an aminoglycoside (Shafran et al 1996; de Lalla et al, 1992; Chiu et al, 1990).

Studies in vitro and in mouse suggest that the limited activity of oral ciprofloxacin alone is related to the inability of ciprofloxacin to achieve bactericidal concentrations at the site of infection (Inderlied et al, 1989; Cipolla et al, 2016); the minimum inhibitory concentration (MIC) of 5 μg/ml versus the clinical serum Cmax of 4 μg/ml explains the limited efficacy in experimental models and in humans (Inderlied et al, 1989). M. abscessus is often resistant to clarithromycin. IV aminoglycosides or imipenem need to be applied, which often are the only available therapeutic alternatives, and these carry the potential for serious side-effects, as well as the trauma and cost associated with IV administration. Clofazimine, linezolid, and cefoxitin are also sometimes prescribed, but toxicity and/or the need for IV administration limit the use of these compounds. Thus, the available therapies have significant deficiencies and improved approaches are needed.

Recent studies also showed that both M. avium and M. abscessus infections are associated with significant biofilm formation (Bermudez et al, 2008; Carter et al, 2003, Nessar at al., 2012): deletion of biofilm-associated genes in M. avium had impact on the ability of the bacterium to form biofilm and to cause pulmonary infection in an experimental animal model (Yamazaki et al, 2006).

Ciprofloxacin is a broad-spectrum fluoroquinolone antibiotic that is active against several other types of gram-negative and gram-positive bacteria and is indicated for oral and IV treatment of lower respiratory tract infections. It acts by inhibition of topoisomerase II (DNA gyrase) and topoisomerase IV, which are enzymes required for bacterial replication, transcription, repair, and recombination. This mechanism of action is different from that for penicillins, cephalosporins, aminoglycosides, macrolides, and tetracyclines, and therefore bacteria resistant to these classes of drugs may be susceptible to ciprofloxacin. There is no known cross-resistance between quinolones—the class of antimicrobials that ciprofloxacin belongs to—and other classes of antimicrobials.

Despite its attractive antimicrobial properties, ciprofloxacin does produce bothersome side effects, such as GI intolerance (vomiting, diarrhea, abdominal discomfort), as well as dizziness, insomnia, irritability and increased levels of anxiety. There is a clear need for improved treatment regimens that can be used chronically, without resulting in these debilitating side effects.

Delivering ciprofloxacin as an inhaled aerosol has the potential to address some of these concerns by compartmentalizing the delivery and action of the drug in the respiratory tract, which is the primary site of infection. Currently there is no aerosolized form of ciprofloxacin with regulatory approval for human use, capable of targeting antibiotic delivery direct to the area of primary infection. In part this is because the poor solubility and bitterness of the drug have inhibited development of a formulation suitable for inhalation; many patients with airway disease may cough or bronchoconstrict when inhaling antibiotics which are not encapsulated in liposomes (Barker et al, 2000). Furthermore, the tissue distribution of ciprofloxacin is so rapid that the drug residence time in the lung is too short to provide additional therapeutic benefit over drug administered by oral or IV routes (Bergogne-Bérézin E, 1993).

The therapeutic properties of many drugs are improved by incorporation into liposomes. Phospholipid vehicles as drug delivery systems were rediscovered as “liposomes” in 1965 (Bangham et al., 1965). The general term “liposome” covers a variety of structures, but all consist of one or more lipid bilayers enclosing an aqueous space in which hydrophilic drugs, such as ciprofloxacin, can be encapsulated. Liposome encapsulation improves biopharmaceutical characteristics through a number of mechanisms including altered drug pharmacokinetics and biodistribution, sustained drug release from the carrier, enhanced delivery to disease sites, and protection of the active drug species from degradation. Liposome formulations of the anticancer agents doxorubicin (Myocet®/Evacet®, Doxyl®/Caelyx®), daunorubicin (DaunoXome®) the anti-fungal agent amphotericin B (Abelcet®, AmBisome®, Amphotec®) and a benzoporphyrin (Visudyne®) are examples of successful products introduced into the US, European and Japanese markets over the last two decades. Recently a liposomal formulation of vincristine (Marqibo®) was approved for an oncology indication. The proven safety and efficacy of lipid-based carriers make them attractive candidates for the formulation of pharmaceuticals.

Therefore, in comparison to the current ciprofloxacin formulations, a liposomal ciprofloxacin aerosol formulation should offer several benefits: 1) higher drug concentrations, 2) increased drug residence time via sustained release at the site of infection, 3) decreased side effects, 4) increased palatability, 5) better penetration into the bacterial biofilms, 6) better penetration into the cells infected by bacteria, and what has been discovered as part of this invention, 7) inhibition of microaggregate formation of NTM

In one example of the current invention, the liposomes encapsulating ciprofloxacin are unilamellar vesicles (average particle size 75-120 nm). Ciprofloxacin is released slowly from these liposomes with a half-life of about 10 hours in the lung (Bruinenberg et al, 2010 b; Cipolla et al, 2016), which allows for once-a-day dosing. Further, studies with a variety of liposome compositions in in vitro and murine infection models showed that liposomal ciprofloxacin is effective against several intracellular pathogens, including M. avium. Inhaled liposomal ciprofloxacin is also effective in treating Pseudomonas aeruginosa (PA) lung infections in patients (Bilton et al, 2009 a, b, 2010, 2011; Bruinenberg et al, 2008, 2009, 2010 a, b, c, d, 2011; Serisier et al, 2013; Cipolla et al, 2016).

Compared to approved doses of oral and IV ciprofloxacin, liposomal ciprofloxacin formulations delivered by inhalation into the airways achieve much greater concentrations in the respiratory tract mucosa and within macrophages with resulting improvement of clinical efficacy: 2 hours post-inhalation of a therapeutic dose of our liposomal ciprofloxacin in patients, the concentration of ciprofloxacin in the sputum exceeded 200 μg/ml, and even 20 hours later (2 hours prior to the next dose), the concentration was >20 μg/ml, well above the minimum inhibitory concentration above for resistant mycobacteria (breakpoint of −4 μg/ml, Bruinenberg 2010b; Cipolla et al, 2016). Since the liposomes containing ciprofloxacin are avidly ingested by macrophages, the ciprofloxacin is brought into close proximity to the intracellular pathogens, thus further increasing anti-mycobacterial concentration and thus should lead to improved efficacy of the inhaled liposomal formulation compared to other forms of ciprofloxacin. We therefore believe that even highly resistant NTM may be suppressed with our inhaled liposomal ciprofloxacin. This is significant because M. avium and M. abscessus resistance to antibiotics is common due to long-term use of systemic antibiotics in these patients.

Our clinical experience with P. aeruginosa (PA) also shows that there is no apparent emergence of resistance following inhaled liposomal ciprofloxacin therapy: in fact, even those patients who also had resistant strains initially, responded well to therapy (Serisier et al., 2013; Cipolla et al, 2016). This is likely due to the presence of sustained overwhelming concentrations of ciprofloxacin. Furthermore, the experience with other anti-pseudomonal drugs tobramycin and colistimethate in patients with cystic fibrosis is that even patients with resistant strains of PA respond clinically well to the inhaled form of the drugs (Fiel, 2008).

Several in vitro studies have demonstrated that liposomal ciprofloxacin is efficacious against intracellular pathogens: 1) In human peripheral blood monocytes/macrophages, liposomal ciprofloxacin tested over concentrations from 0.1 to 5 μg/ml caused concentration-related reductions in intracellular M. avium-M. intracellulare complex (MAC) colony forming units (CFU) compared to free drug at the same concentrations (Majumdar et al, 1992); 2) In a murine macrophage-like cell line J774, liposomal ciprofloxacin decreased the levels of cell associated M. avium up to 43-fold and these reductions were greater than for free ciprofloxacin (Oh et al, 1995).

Once M. avium or M. abscessus infect monocytes/macrophages, the infection can then spread to the lungs, liver, spleen, lymph nodes, bone marrow, and blood. There are no published studies on the efficacy of liposomal ciprofloxacin against M. avium or M. abscessus in animal models.

A few in vivo studies have demonstrated that liposomal ciprofloxacin is efficacious against the intracellular pathogen, F. tularensis: Efficacy of liposomal ciprofloxacin delivered to the lungs by inhalation or intranasal instillation against inhalational tularemia (F. tularensis live vaccine strain (LVS) and Schu S4) in mice, was demonstrated with as little as a single dose of liposomal ciprofloxacin providing 100% protection post-exposure, and even effective post-exposure treatment for animals that already had significant systemic infection (Blanchard et al, 2006; Di Ninno et al, 1993; Conley et al, 1997; Hamblin et al, 2011; Hamblin et al, 2014; Wong et al, 2003). These studies also found that inhaled liposomal ciprofloxacin was superior to both inhaled and oral unencapsulated ciprofloxacin.

In contrast, a) free ciprofloxacin was inferior to liposomal ciprofloxacin in macrophage models of mycobacterial infections (Majumdar et al, 1992; Oh et al, 1995); b) free ciprofloxacin alone delivered to the lungs had inferior efficacy to free ciprofloxacin when tested in murine models of F. tularensis infection (Conley et al, 1997; Wong et al, 2003), as it is rapidly absorbed into the blood stream. A formulation made up of both free and liposomal ciprofloxacin combines the potential advantages of an initial transient high concentration of free ciprofloxacin to increase Cmax in the lungs, followed by the slow release of ciprofloxacin from the liposomal component, as demonstrated in non-CF bronchiectasis patients by Aradigm (e.g., Cipolla et al, 2011; Serisier et al, 2013; Cipolla et al, 2016). The free ciprofloxacin component also has a desirable immunomodulatory effect (U.S. Pat. Nos. 8,071,127, 8,119,156, 8,268,347 and 8,414,915).

Further, liposomal ciprofloxacin injected parenterally activates macrophages, resulting in increased phagocytosis, nitric oxide production, and intracellular microbial killing even at sub-inhibitory concentrations, perhaps via immunostimulatory effects (Wong et al, 2000). The ciprofloxacin-loaded macrophages may migrate from the lungs into the lymphatics to treat infections in the liver, spleen, and bone marrow—as suggested by the systemic effects of pulmonary-delivered CFI in tularemia (e.g., Di Ninno et al, 1993; Conley et al, 1997; Hamblin et al, 2011; Hamblin et al, 2014; Wong et al, 2003). Liposome-encapsulated antibiotics are also known to better penetrate bacterial films formed by P. aeruginosa in the lungs (e.g., Meers et al, 2008).

Recently it was demonstrated that liposomally encapsulated ciprofloxacin would inhibit the biofilm formation of both M. avium and M. abscessus and microaggregates of M. avium including inhibiting the gene expression of MAV_3013 and MAV_0831 on which the formation of M. avium microaggregate in vivo is dependent (Blanchard et al., 2014; Bermudez et al., 2016). The anti-mycobacterial and immunomodulatory effects of these formulations may provide better alternatives to the existing treatments for patients infected with M. avium or M. abscessus, or provide an adjunct for incremental improvements if the antibiotic preparation is effective against these organisms that are planktonic, as well as in the biofilms and within macrophages. It is further required that the antibiotic treatment is well tolerated and safe when given by inhalation. Since the current antibiotic treatment options often cause serious systemic side-effects, it is desirable for the new treatment to have less toxic antibiotics and to minimize their concentration in the circulation to avoid systemic side effects.

A previous study of liposomal ciprofloxacin demonstrated high uptake by alveolar macrophages in animals, which is presumably the reason for the highly effective post-exposure prophylaxis and treatment of inhalational tularemia in mice. Although the plasma levels of ciprofloxacin were low following respiratory tract administration of our liposomal ciprofloxacin, a reduction of the tularemia infection from the liver, spleen, tracheobronchial lymph nodes, as well as the lungs, was observed suggesting that the alveolar macrophages loaded with liposomal ciprofloxacin migrate from the lungs via lymph into the liver, spleen and lymph nodes (F. tularensis CFU levels in bone marrow and blood were not measured) (Conley et al, 1997).

### SUMMARY OF THE INVENTION

In accordance with the inventive patients who are susceptible to NTM infections are identified prior to NTM microaggregate formation, which leads to infection and biofilm formation are treated with inhaled therapy to prevent formation of NTM aggregates and thus formation of biofilm. Thereby, improving patient outcome as compared to the current paradigm.

Treatment is carried out with a formulation of inhaled liposomal ciprofloxacin or combinations of unencapsulated ciprofloxacin and liposomal ciprofloxacin, to prevent NTM microaggregate formation and thus inhibit biofilm formation. Patients susceptible to NTM infections are treated by once-daily inhalation with the formulation, or more frequently if desirable, which could also be combined with other treatments if needed. The target patient population includes patients with a prior history of NTM infections, or infections with other pathogens in the lungs or airways.

Liposomes are used to improved penetration of drugs into bacterial biofilms wherein the liposomes are phagocytosed by infected macrophages in general (Meers et al, 2008). Encapsulation of antibiotics including fluoroquinolones and aminoglycosides has been demonstrated (Finlay and Wong, 1998; Cipolla et al, 2016; Meers et al, 2008) both liposomal ciprofloxacin and liposomal amikacin have been shown to be effective against NTM as well as against P. aeruginosa (Olivier et al., 2017; Serisier et al., 2013).

The combination of the encapsulation of antibiotics in liposomes with direct delivery of the formulation to the lungs makes these treatments fundamentally different from oral and parenteral products of antibiotics in terms of biodistribution, pharmacokinetics, as well as improved safety and efficacy (Cipolla et al, 2016). For example, the liposome-encapsulated ciprofloxacin is delivered at very high concentrations directly to the respiratory tract where it resides over a prolonged period of time, during which ciprofloxacin is slowly released from the liposomes to the site of infection in the lung, and with lower systemic exposure compared to oral or IV ciprofloxacin (Cipolla et al, 2016).

The size and composition of the liposomal ciprofloxacin formulations are also designed to facilitate uptake by the macrophages in the lung. An important feature is that the formulation should be robust to the nebulization process so that the liposomes retain their size and encapsulation characteristics. If the liposomes are not robust to aerosolization, then there could be loss of encapsulated drug, or a change in the liposome size or surface characteristics (Cipolla et al, 2010, 2013a, 2013b). Either of these changes, or others that have not been described, might lead to a change in the release profile and thus the antibiotic concentration in the airways relative to the efficacious concentration, and a lower uptake of the liposomes by macrophages which can harbor intracellular infections including NTM. The presence of the liposomes may also be a contributing factor to efficacy, as the data in the examples described below show that the free drug alone was not efficacious and required the liposomal component. The liposomes that lose a portion of their encapsulated drug during nebulization or aerosolization, even if they are taken up by the macrophages with the same efficiency as uncompromised liposomes, now have less encapsulated drug and thus a lower payload to treat the infectious agent inside the macrophages and in biofilms. This modification has the potential to reduce the efficacy of treatment to prevent formation of NTM microaggregates whether on the airway surface or elsewhere in the lung.

One particular composition of liposomes, which are covered by this invention, are relatively uncompromised by the nebulization process and have been described in U.S. Pat. Nos. 8,071,127, 8,119,156, 8,268,347 and 8,414,915. Those patents describe an aerosolizable formulation producing inhaled droplets or particles with bi-phasic release of antibiotic. The droplets or particles comprise a free drug (e.g., an anti-infective compound) in which drug is not encapsulated and which may be ciprofloxacin. The particles further comprise a liposome which encapsulates a drug such as an anti-infective compound which also may be ciprofloxacin. The free and liposome encapsulated drug are included within a pharmaceutically acceptable excipient which is formulated for aerosolized delivery. The particles may further include an additional therapeutic agent which may be free and/or in a liposome and which can be any pharmaceutically active drug which is different from the first drug.

Other liposome compositions include those which are modified by nebulization, leading to changes in vesicle size, or drug encapsulation, or both (Cipolla et al, 2013a and Cipolla et al, 2013b). These include formulations of liposomal ciprofloxacin which are not robust to the nebulization process (Finlay and Wong, 1998). These include liposomes containing drugs such as amikacin that have been described in U.S. Pat. Nos. 8,226,975, 8,642,075, 8.673.348, 8,673,349, and U.S. Patent applications: 2007196461, 20130028960, 20130052260, 20130064883, 20130071469, 20130087480, 20130330400, 20140072620. US Patent application 20130330400 specifically describes a liposomal formulation of amikacin that is compromised by nebulization such that only 58 to 73% of the drug remains encapsulated after exposure to nebulization. In this application, US Patent application 20130330400, the mean vesicle size was also affected by the nebulization process with a reduction from a mean of 285 nm prior to nebulization to 265 nm after nebulization (range: 249 to 289 nm). US Patent application 20140072620 also describes a liposomal amikacin formulation that degrades to 60% encapsulated and 40% free drug after nebulization.

An example of liposomes of our invention retains 80% or more, and preferably 90% or more, and most preferably 95% or more of the encapsulated drug after nebulization relative to that which was encapsulated prior to nebulization (Cipolla et al, 2010, Cipolla et al, 2013b). If significant amounts of the drug are lost from the liposomes during nebulization, for example, greater than 20% of the encapsulated drug, then the liposomes will not contain as much antibiotic and so may not be as effective at inhibiting NTM microaggregate formation and the formation of biofilm. Another component is that retention of drug encapsulation following nebulization ensures that more drug remains within the liposomes that are taken up by macrophages, often the site of NTM infection and biofilm formation.

Aerosol delivery of liposomal antibiotics may be preferable if the ratio of the encapsulated to unencapsulated drug delivered to the patients' lungs is predictable. This can be achieved by judicious choice of the formulation as well as by selection of a suitable aerosolization equipment. For example, to prevent the formation of bacterial biofilms, it may be preferable to have a high percentage of encapsulation. A large concentration of unencapsulated antibiotic may be preferable if the bacterial infection that is targeted for the treatment responds to high peaks rather than sustained concentrations, or such property is preferred for safety reasons. The alveolar macrophages are targeted by M. avium and M. abscessus (Jordao et al, 2008) and other mycobacteria species as well. The macrophages avidly ingest both the liposomal ciprofloxacin and the mycobacteria, bringing both into close proximity within the phagosomes. This increase in the bioavailability at the infected target, the surface of the airways and the alveolar macrophage cells in the lung, should lead to improved efficacy versus systemically delivered ciprofloxacin or other anti-mycobacterial agents. The sustained-release of ciprofloxacin from the liposomes further increases the ratio of the area under the curve to MIC (AUC/MIC) in the lungs and macrophages, in particular, and may enable once-a-day dosing. The administration of these formulations will likely cause a lower incidence of relapse and adverse systemic effects.

An aspect of the invention is an aerosol of inhaled droplets or particles. The droplets or particles comprise a free drug (e.g., an anti-infective compound) in which drug is not encapsulated and which may be ciprofloxacin. The particles further comprise a liposome which encapsulates a drug such as an anti-infective compound which also may be ciprofloxacin. The free and liposome encapsulated drug are included within a pharmaceutically acceptable excipient which is formulated for aerosolized delivery. The particles may further include an additional therapeutic agent which may be free and/or in a liposome and which can be any pharmaceutically active drug which is different from the first drug.

Another aspect of the invention is a formulation comprising liposomes which are delivered via an aerosol to the respiratory tract of a human patient or an infected animal with an NTM infection, or to prevent an NTM infection, the liposomes comprising free and encapsulated ciprofloxacin. The liposomes may be unilamellar or multilamellar. The aerosolization can be achieved by nebulization, including jet nebulization or mesh nebulization. The encapsulated ciprofloxacin is in liposomes which are robust to the nebulization process and maintain their encapsulation state to greater than 80% following nebulization, preferably greater than 90% following nebulization, and more preferably to greater than 95% following nebulization.

A third aspect of the invention is a method for preventing or treating intracellular infections in a patient, the method comprising administering a formulation comprising the anti-infective; e.g., ciprofloxacin, encapsulated in liposomes to the patient. The formulation is preferably administered by inhalation to the patient, and more preferably by nebulization. The intracellular infections may represent NTM infections including M. abscessus, M. avium, M. avium complex, (MAC) (M. avium and M. intracellulare), M. Bolletii, M. chelonae, M. ulcerans, M. xenopi, M. kansasii, M. fortuitum complex (M. fortuitum and M. chelonae) or M. marinum infections.

A fourth aspect to the invention is the ability of the liposomal anti-infective formulation, preferably a liposomal ciprofloxacin formulation, after aerosolization and delivery to the respiratory tract of a human or animal, to prevent, inhibit, or reduce microaggregate formation, either on the surface of the airways or elsewhere within the lung.

The fifth aspect of the invention is that for the treatment to be maximally effective, the antibiotic formulation also needs to be able to penetrate the biofilm formed by the mycobacteria.

The sixth aspect of the invention is that the antibiotic in a suitable vehicle is not only able to penetrate the biofilm but also to have efficacy against both sessile (dormant) and replicating mycobacteria.

A seventh aspect of the invention is that the antibiotic inhibits the formation of mycobacterial biofilms in the lung. McNabe et al. (2012) state that in particular, M. avium forms biofilm, a property in mice that is associated with lung infection via aerosol. In their studies, they found that incubation of M. avium with two antibiotics found in the environment, streptomycin and tetracycline, resulted in an increase, not decrease, in the biofilm formation. Other antibiotics, including ampicillin, moxifloxacin, rifampicin and TMP/SMX had no effect on biofilm; i.e., they were not able to kill the M. avium. Moxifloxacin is a fluoroquinolone, like ciprofloxacin, so it is indeed surprising that we have found that specific liposomal ciprofloxacin formulations are effective at killing mycobacteria in biofilm. Note that even if an antibiotic is able to kill all of the planktonic phenotype of mycobacteria, both planktonic and sessile bacteria are able to establish infection equally, ensuring that the remaining sessile bacteria will reinfect the host (McNabe et al. 2012). McNabe et al go on to state that that many patients with chronic lung conditions are treated for infections caused by many pathogens with antibiotics, such as aminoglycosides or tetracyclines. Therefore, there is a possibility that, in the situation that M. avium is colonizing an individual receiving an antibiotic, either for prophylaxis or therapy, it would potentially result in the production of increased amounts of biofilm and further establishment of the infection (McNabe et al, 2012).

An eighth aspect of the present invention is a formulation comprising both a free and encapsulated anti-infective providing an initially high therapeutic level of the anti-infective in the lungs, while maintaining a sustained release of anti-infective over time, to overcome the barrier to eradicate the difficult to treat biofilm bacteria. The intent of the immediate-release anti-infective; e.g., ciprofloxacin, is thus to rapidly increase the antibiotic concentration in the lung to therapeutic levels above the MIC. The sustained-release anti-infective; e.g., ciprofloxacin, serves to maintain a therapeutic level of antibiotic in the lung thereby providing continued therapy over a longer time frame, increasing efficacy, reducing the frequency of administration, and reducing the potential for microaggregates of NTM to form. The sustained release of the anti-infective may ensure that the anti-infective agent never falls below the sub-inhibitory concentration and so reduces the likelihood of forming resistance to the anti-infective.

The liposomes described in the pharmaceutical formulations of the present invention can be comprised of lipids or sterols or combinations of lipids and sterols. In particular, the compositions of the formulations can include dipalmitoylphosphatidylcholine (DPPC), a major constituent of naturally-occurring lung surfactant, or hydrogenated soy phosphatidylglycerol (HSPC) as has been described in the examples below. Other lipids can be used in the formulations described in this invention. The lipids may be synthetic, semi-synthetic or naturally-occurring lipids, including phospholipids, tocopherols, sterols, fatty acids, glycoproteins such as albumin, negatively-charged lipids and cationic lipids. In terms of phospholipids, they could include such lipids as egg phosphatidylcholine (EPC), egg phosphatidylglycerol (EPG), egg phosphatidyl-inositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. Other examples include dimyristoylphosphatidycholine (DMPC) and dimyristoylphospha-tidylglycerol (DMPG), dipalmitoylphosphatidylcholine (DPPC) and dipalmitoyl-phosphatidylglycerol (DPPG), distearoylphospha-tidylcholine (DSPC) and distearoylphosphatidylglycerol (DSPG), dioleylphospha-tidylethanolamine (DOPE) and mixed phospholipids like palmitoylstearoyl-phosphatidylcholine (PSPC) and palmitoylstearolphosphatidylglycerol (PSPG), and single acylated phospholipids like mono-oleoyl-phosphatidylethanolamine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

The liposomes are comprised of particles with a mean diameter of approximately 10 nanometers to approximately 5.0 microns, preferably in the range about 50 to 300 nanometers. The sustained release property of the liposomal product can be regulated by the nature of the lipid membrane and by inclusion of other excipients (e.g., sterols) in the composition.

Although ciprofloxacin is a particularly useful anti-infective in this invention, there is no desire to limit this invention to ciprofloxacin. Other antibiotics or anti-infectives can be used such as those selected from the group consisting of: an aminoglycoside (e.g., amikacin or tobramycin), a tetracycline, a sulfonamide, p-aminobenzoic acid, a diaminopyrimidine, a quinolone, a beta-lactam, a beta-lactam and a beta-lactamase inhibitor, chloramphenicol, a macrolide, penicillins, cephalosporins, linomycin, clindamycin, spectinomycin, polymyxin B, colistin, vancomycin, bacitracin, isoniazid, rifampin, ethambutol, ethionamide, aminosalicylic acid, cycloserine, capreomycin, a sulfone, clofazimine, thalidomide, a polyene antifungal, flucytosine, imidazole, triazole, griseofulvin, terconazole, butoconazole ciclopirax, ciclopirox olamine, haloprogin, tolnaftate, naftifine, terbinafine, or any combination thereof.

Antibiotics that are effective against formation of NTM microaggregates are preferred.

These and other objects, advantages, and features of the invention will become apparent to those persons skilled in the art upon reading the details of the formulations and methodology as more fully described below.

### BRIEF DESCRIPTION OF THE DRAWINGS

Aspects and embodiments of the invention are best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

The growth of the microaggregates of M. avium over 24 h on the epithelial cell monolayer for the experimental conditions shown for Example 3 (Table 7), i.e., for treatment with CFI and free ciprofloxacin (300 μg/mL) is shown the electron micrographs in FIG. 1.

FIG. 1 consists of FIGS. 1A, 1B, 1C, 1D and 1E. Electron Micrographs of M. avium Microaggregates: Figure (A) shows control added at the same time as the bacteria (t=0 h); the bacterial microaggregates are visible. Figure (B) shows free ciprofloxacin treatment added at t=0 h, which has much less microaggregate formation than control. Figures (C) and (D) show CFI treatment added at t=0 h before aggregate formation. Aggregation is prevented and very little microaggregate is present. Figure (E) shows CFI treatment added at t=24 h to already present microaggregates. The bacterial surface is unusual, i.e., possibly due to liposomes on the surface or the effect of ciprofloxacin on the viability of the bacteria.

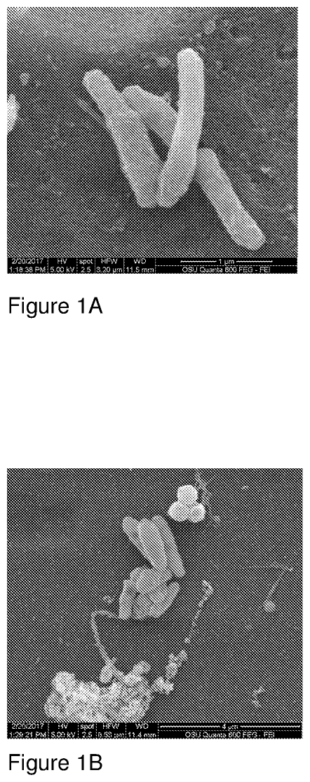

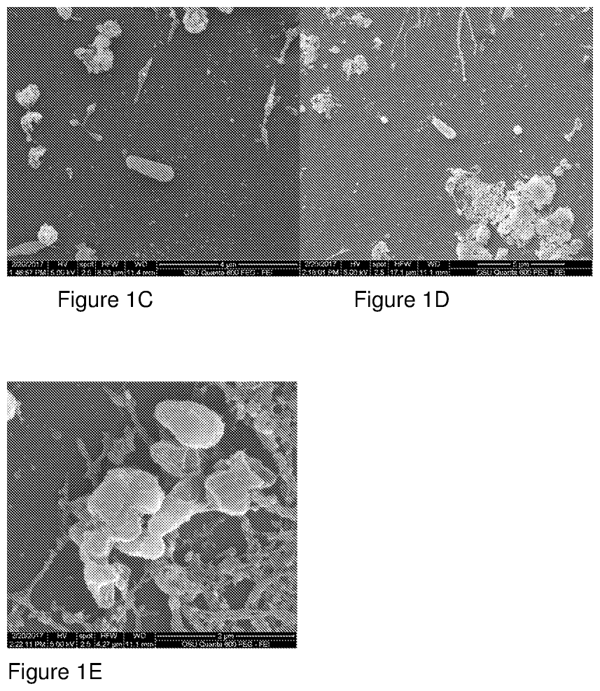

### DETAILED DESCRIPTION OF THE INVENTION

Before the present method of formulating ciprofloxacin-encapsulated liposomes and delivery of such for prevention and/or treatment of NTM infections and other medical conditions, and devices and formulations used in connection with such are described, it is to be understood that this invention is not limited to the particular methodology, antibiotic choice, devices and formulations described, as such methods, devices, antibiotics and formulations may, of course, vary. It is also to be understood that the terminology used herein is for the purpose of describing particular embodiments only, and is not intended to limit the scope of the present invention which will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, anti-infective refers to agents that act against infections, such as bacterial, viral, fungal, mycobacterial, or protozoal infections.

Anti-infectives covered by the invention include but are not limited to quinolones (such as nalidixic acid, cinoxacin, ciprofloxacin, levofloxacin, sparfloxacin, trovafloxacin, oxolinic acid, grepafloxacin, ofloxacin, lomofloxacin, moxifloxacin, enoxacin and norfloxacin and the like), sulfonamides (e.g., sulfanilamide, sulfadiazine, sulfamethaoxazole, sulfisoxazole, sulfacetamide, and the like), aminoglycosides (e.g., streptomycin, gentamicin, tobramycin, amikacin, netilmicin, kanamycin, and the like), tetracyclines (such as chlortetracycline, oxytetracycline, methacycline, doxycycline, minocycline and the like), para-aminobenzoic acid, diaminopyrimidines (such as trimethoprim, often used in conjunction with sulfamethoxazole, pyrazinamide, and the like), penicillins (such as penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (such as methicillin, oxacillin, cloxacillin, dicloxacillin, nafcillin and the like), first generation cephalosporins (such as cefadroxil, cephalexin, cephradine, cephalothin, cephapirin, cefazolin, and the like), second generation cephalosporins (such as cefaclor, cefamandole, cefonicid, cefoxitin, cefotetan, cefuroxime, cefuroxime axetil, cefinetazole, cefprozil, loracarbef, ceforanide, and the like), third generation cephalosporins (such as cefepime, cefoperazone, cefotaxime, ceftizoxime, ceftriaxone, ceftazidime, cefixime, cefpodoxime, ceftibuten, and the like), other beta-lactams (such as imipenem, meropenem, aztreonam, clavulanic acid, sulbactam, tazobactam, and the like), beta-lactamase inhibitors (such as clavulanic acid), chloramphenicol, macrolides (such as erythromycin, azithromycin, clarithromycin, and the like), lincomycin, clindamycin, spectinomycin, polymyxin B, polymixins (such as polymyxin A, B, C, D, E.sub.1(colistin A), or E.sub.2, colistin B or C, and the like) colistin, vancomycin, bacitracin, isoniazid, rifampin, ethambutol, ethionamide, aminosalicylic acid, cycloserine, capreomycin, sulfones (such as dapsone, sulfoxone sodium, and the like), clofazimine, thalidomide, or any other antibacterial agent that can be lipid encapsulated. Anti-infectives can include antifungal agents, including polyene antifungals (such as amphotericin B, nystatin, natamycin, and the like), flucytosine, imidazoles (such as miconazole, clotrimazole, econazole, ketoconazole, and the like), triazoles (such as itraconazole, fluconazole, and the like), griseofulvin, terconazole, butoconazole ciclopirax, ciclopirox olamine, haloprogin, tolnaftate, naftifine, terbinafine, or any other antifungal that can be lipid encapsulated or complexed and pharmaceutically acceptable salts thereof and combinations thereof. Discussion and the examples are directed primarily toward ciprofloxacin but the scope of the application is not intended to be limited to this anti-infective. Combinations of drugs can be used.

As used herein, “Formulation” refers to the liposome-encapsulated anti-infective, with any excipients or additional active ingredients, either as a dry powder or suspended or dissolved in a liquid.

The terms “subject,” “individual,” “patient,” and “host” are used interchangeably herein and refer to any vertebrate, particularly any mammal and most particularly including human subjects, farm animals, and mammalian pets. The subject may be, but is not necessarily under the care of a health care professional such as a doctor.

A “stable” formulation is one in which the active ingredient therein essentially retains its physical and chemical stability and integrity upon storage and exposure to relatively high temperatures or other stress such as shaking, shipping, dropping or handling. Various analytical techniques for measuring the stability of the active ingredient are available in the art. Stability can be measured at a selected temperature for a selected time period.

“Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the claimed methods and compositions.

### Invention in General

Ciprofloxacin is a well-established and extensively utilized broad-spectrum fluoroquinolone antibiotic that is indicated for the treatment of lower respiratory tract infections, due to, for example, P. aeruginosa, which is common in patients with cystic fibrosis. The primary advantage of inhaled antimicrobials is that they target antibiotic delivery to the area of primary infection and bypass GI-related side effects; however, the poor solubility and bitterness of the drug have limited development of a formulation suitable for inhalation. Furthermore, the rapid tissue distribution of ciprofloxacin means a short drug residence time in the lung thus limiting therapeutic benefit over oral or IV drug administration. The liposome-encapsulated formulations of ciprofloxacin described here decrease the limitations and improves management of pulmonary infections due to NTM through improved biopharmaceutical characteristics and mechanisms such as retention of vesicle size and encapsulation following nebulization, altered drug PK and biodistribution, sustained drug release from the carrier, enhanced delivery to disease sites including intracellular infections, whereby the concentration of drug is now higher within the intracellular space.

The invention is not limited to the treatment of patients with a prior history or current history of NTM infections or other infectious agents. In fact, there are many patients and indications for which this therapy may be beneficial, including those who are suspected of harboring, or with the potential to harbor, intracellular infections and particularly those infections in alveolar macrophages and/or biofilms in the airways. However, it is particularly useful against mycobacterial infections because it is effective at preventing the formation of microaggregates of NTM, as well as killing both replicating and non-replicating bacteria, which are present in biofilm. As described by McNabe et al (2012), M. avium forms increasing amounts of biofilm in presence of antibiotics such as streptomycin and tetracycline, which stimulate biofilm-related gene expression in the bacterium. Once formed, biofilms are made of two distinct populations of bacteria, sessile, the more resistant phenotype, and planktonic, a susceptible phenotype. This it is indeed surprising that inhaled liposomal ciprofloxacin is effective at killing both populations of bacteria, including sessile, which are more resistant. This should be contrasted to a much weaker efficacy of unencapsulated ciprofloxacin. The difference between liposomal and encapsulated ciprofloxacin activity against NTM would be likely to be even greater in vivo because the unencapsulated ciprofloxacin disappears from the airways and the lung much faster than the encapsulated ciprofloxacin.

The formulations of this invention may include liposomal ciprofloxacin, generally referred to as Ciprofloxacin for Inhalation (CFI), and combinations of CFI and free ciprofloxacin, generally termed Pulmaquin or dual release ciprofloxacin for inhalation).

The formulations of the invention may be administered to a patient using a disposable package and portable, hand-held, battery-powered device, such as the AERx device (U.S. Pat. No. 5,823,178, Aradigm, Hayward, Calif.). Alternatively, the formulations of the instant invention may be carried out using a mechanical (non-electronic) device. Other inhalation devices may be used to deliver the formulations including conventional jet nebulizers, ultrasonic nebulizers, soft mist inhalers, dry powder inhalers (DPIs), metered dose inhalers (MDIs), condensation aerosol generators, and other systems. The proportion of free ciprofloxacin to encapsulated ciprofloxacin was shown to remain constant after nebulization; i.e., there was no damage to the liposomes during nebulization that would result in premature release of a portion of the encapsulated antibiotic. This finding is unexpected based upon prior literature reports (Niven R W and Schreier H, 1990) but ensures that the animal or human inhaling the aerosol will get a reproducible proportion of free to encapsulated drug depositing throughout the lung.

An aerosol may be created by forcing drug through pores of a membrane which pores have a size in the range of about 0.25 to 6 microns (U.S. Pat. No. 5,823,178). When the pores have this size the particles which escape through the pores to create the aerosol will have a diameter in the range of 0.5 to 12 microns. Drug particles may be released with an air flow intended to keep the particles within this size range. The creation of small particles may be facilitated by the use of the vibration device which provides a vibration frequency in the range of about 800 to about 4000 kilohertz. Those skilled in the art will recognize that some adjustments can be made in the parameters such as the size of the pores from which drug is released, vibration frequency, pressure, and other parameters based on the density and viscosity of the formulation keeping in mind that an object of some embodiments is to provide aerosolized particles having a diameter in the range of about 0.5 to 12 microns.

The liposome formulation may be a low viscosity liquid formulation. The viscosity of the drug by itself or in combination with a carrier should be sufficiently low so that the formulation can be forced out of openings to form an aerosol, e.g., using 20 to 200 psi to form an aerosol preferably having a particle size in the range of about 0.5 to 12 microns.

In an embodiment, a low boiling point, highly volatile propellant is combined with the liposomes of the invention and a pharmaceutically acceptable excipient. The liposomes may be provided as a suspension or dry powder in the propellant, or, in another embodiment, the liposomes are dissolved in solution within the propellant. Both of these formulations may be readily included within a container which has a valve as its only opening. Since the propellant is highly volatile, i.e. has a low boiling point, the contents of the container will be under pressure.

In accordance with another formulation, the ciprofloxacin-containing liposomes are provided as a dry powder by itself, and in accordance with still another formulation, the ciprofloxacin-containing liposomes are provided in a solution formulation. The dry powder may be directly inhaled by allowing inhalation only at the same measured inspiratory flow rate and inspiratory volume for each delivery. The powder may be dissolved in an aqueous solvent to create a solution which is moved through a porous membrane to create an aerosol for inhalation. Any formulation which makes it possible to produce aerosolized forms of ciprofloxacin-containing liposomes which can be inhaled and delivered to a patient via the intrapulmonary route may be used in connection with the present invention. Specific information regarding formulations which can be used in connection with aerosolized delivery devices are described within Remington's Pharmaceutical Sciences, A. R. Gennaro editor (latest edition) Mack Publishing Company. Regarding insulin formulations, it is also useful to note the findings of Sciarra et al., (1976). When low boiling point propellants are used, the propellants are held within a pressurized canister of the device and maintained in a liquid state. When the valve is actuated, the propellant is released and forces the active ingredient from the canister along with the propellant. The propellant will “flash” upon exposure to the surrounding atmosphere, i.e., the propellant immediately evaporates. The flashing occurs so rapidly that it is essentially pure active ingredient which is actually delivered to the lungs of the patient.

Based on the above, it will be understood by those skilled in the art that a plurality of different treatments and means of administration can be used to treat a single patient. Thus, patients already receiving such medications, for example, as intravenous ciprofloxacin or antibiotics, etc., may benefit from inhalation of the formulations of the present invention. Some patients may receive only ciprofloxacin-containing liposome formulations by inhalation. Such patients may be diagnosed as having NTM lung infections, or have symptoms of a medical condition, which symptoms may benefit from administration to the patient of an antibiotic such as ciprofloxacin. The formulations of the invention may also be used diagnostically.

A patient will typically receive a dose of about 0.01 to 10 mg/kg/day of ciprofloxacin ±20% or ±10%. This dose will typically be administered by at least one, preferably several “puffs” from the aerosol device. The total dose per day is preferably administered at least once per day, but may be divided into two or more doses per day. Some patients may benefit from a period of “loading” the patient with ciprofloxacin with a higher dose or more frequent administration over a period of days or weeks, followed by a reduced or maintenance dose. As NTM is a difficult condition to treat, patients are expected to receive such therapy over a prolonged period of time.

### EXPERIMENTAL

The following examples are put forth so as to provide those of ordinary skill in the art with a complete disclosure and description of how to make and use the present invention, and are not intended to limit the scope of what the inventors regard as their invention nor is it intended to represent that the experiment below is the only experiment performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Formulations of Liposomal Ciprofloxacin (CFI), Free Ciprofloxacin (FCI), Combinations or Mixtures of CFI and FCI to Produce Pulmaquin, or Dual Release Ciprofloxacin for Inhalation (DRCFI), and Liposomes Containing Encapsulated Ciprofloxacin in the Form of Nanocrystals (Nanocrystal):

Ciprofloxacin HCl (50 mg/mL), or ciprofloxacin in the base form (45 mg/mL), is encapsulated into liposomes consisting of hydrogenated soy phosphatidylcholine (HSPC) (approximately 60 to 70 mg/mL), a semi-synthetic fully hydrogenated derivative of natural soy lecithin (HSPC), and cholesterol (approximately 25 to 30 mg/mL). The lipid is organized in a bilayer, with an average particle size of 75 to 120 nm. The sterile suspension is suspended in an isotonic buffer (25 mM histidine, 145 mM NaCl at pH 6.0, 300 mOsm/kg) and administered by inhalation. These liposomal ciprofloxacin formulations contain approximately 1% unencapsulated ciprofloxacin but can be combined with free ciprofloxacin (10 to 30 mg/mL as the hydrochloride salt or 8 to 27 mg/mL as ciprofloxacin base) in solution. It is possible to adjust the ratio of free and liposomally encapsulated ciprofloxacin in any ratio and to dilute or concentrate the formulations. Liposomes containing nanocrystalline ciprofloxacin were produced as described in the patent application of Cipolla et al (U.S. Patent application 2015/0283076).

Liposomes can be produced by a variety of methods known in the art. Techniques for producing large unilamellar vesicles (LUVs), such as, reverse phase evaporation, infusion procedures, and detergent dilution, can be used to produce liposomes. A review of these and other methods for producing liposomes may be found in the three volume text: Liposome Technology (Third Edition, edited by Gregory Gregoriadis). Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization can also be so used to produce smaller unilamellar liposomes from larger liposomes.

### Example 2

### Activity of Liposomal Ciprofloxacin Against M. avium in Human Macrophage Model (Blanchard et al., 2014)

Rationale: Individuals with chronic lung pathology such as bronchiectasis, emphysema and cystic fibrosis frequently develop pulmonary infection caused by M. avium. The infection is characterized in the majority of the patients as peri-bronchiolar, with the development of granulomas. Treatment with the current recommended antibiotics is often insufficient to cure the condition. The efficacy of liposome-ciprofloxacin delivered by the respiratory route was evaluated.

Methods: Human macrophage (THP-1) monolayers were established and then the cells were infected with M. avium strain 101 or 109, which was done by exposing the macrophages to the bacteria for 1 hour and then allowing the bacteria to replicate intracellularly for 18 hours. The infected macrophages were then treated with 20 μg/ml of either free ciprofloxacin, CFI, or nanocrystalline ciprofloxacin (Nanocrystal) for 4 days and then the number of viable intracellular bacteria were quantified.

Results: Table 1 shows the colonization of M. avium 101 or M. avium 109 for each arm. Treatment of 20 μg/ml with CFI or liposomes containing nanocrystalline ciprofloxacin (Nanocrystal) were found to provide a statistically significant effect in each of these models versus the initial infecting load (CFU) in macrophages on Day 0. Specifically, for M. avium 101, both CFI and Nanocrystal significantly decreased (p<0.05) CFU by 88% and 86%, respectively. Similarly, for M. avium 109, both CFI and Nanocrystal significantly decreased (p<0.05) CFU by 72% and 47%, respectively. However, free ciprofloxacin alone did not have a statistically significant effect.

TABLE 1
Activity of FCI and Ciprofloxacin-liposome formulations
at 20 μg/mL against M avium in Macrophages

| Bacterial | | CFU | |
|---|---|---|---|
| strain | Treatment | Day 0 | Day 4 |
| M. avium 101 | Buffer control | 7 ± 0.2 × 10⁴ | 5.5 ± 0.2 × 10⁵ |
| | Ciprofloxacin | 7 ± 0.2 × 10⁴ | 4.9 ± 0.4 × 10⁵ |
| | CFI | 7 ± 0.2 × 10⁴ | 8.1 ± 0.3 × 10³ * |
| | Nanocrystal | 7 ± 0.2 × 10⁴ | 9.6 ± 0.4 × 10³ * |
| M. avium 109 | Buffer control | 1.4 ± 0.6 × 10⁴ | 6.0 ± 0.5 × 10⁴ |
| | Ciprofloxacin | 1.4 ± 0.4 × 10⁴ | 1.1 ± 0.4 × 10⁴ |
| | CFI | 1.4 ± 0.4 × 10⁴ | 3.9 ± 0.5 × 10³ * |
| | Nanocrystal | 1.4 ± 0.6 × 10⁴ | 7.4 ± 0.3 × 10³ * |

* p < 0.05 compared to the initial infecting load (CFU) in macrophages on Day 0

### Example 3

### Inhibition of Gene Expression for M. avium Microaggregate Formation (Bermudez et al., 2016)

Introduction: M. avium is an important pathogen in individuals with immunosuppression as well as with underlying lung pathology such as cystic fibrosis, bronchiectasis and emphysema. In such patients with chronic lung conditions, M. avium causes debilitating diseases, requiring long courses of therapy, frequently with side-effects and therapeutic failure. We evaluated whether liposomal ciprofloxacin (CFI) would affect gene expression of MAV-3013 and MAV-0831 and thus the establishment of M. avium microaggregates, the first step for biofilm formation in the lung airways. CFI has shown efficacy for the treatment of M. avium and M. abscessus infections in vitro and in vivo.

Methods: M. avium strains 104 and A5 are clinical isolates; both strains form robust biofilms in vitro and in vivo. Biofilm and microaggregates of M. avium were developed. CFI and ciprofloxacin were tested at 15 and 300 mg/ml (concentrations encountered in treated lungs in humans with CFI).

Results: CFI significantly decreased gene expression of MAV_3013 and MAV_0831 at both 15 and 300 μg/mL; CFI at 15 μg/mL had significantly greater effect on microaggregate-associated genes than in bacterial viability.

TABLE 2
Gene expression (qRT-PCR): MAV-3013 and MAV-0831, associated
with microaggregate formation measured at t = 1 h
(Antibiotic added concomitant to bacteria at t = 0 h)

| | Hsp65 | MAV-3013 | MAV-0831 |
|---|---|---|---|
| No antibiotic | 6.2 ± 0.3 | 8.4 ± 0.7 | 6.1 ± 0.4 |
| CFI (15 μg/ml) | 5.9 ± 0.8 | 3.3 ± 0.5 | 2.7 ± 0.6 |
| Ciprofloxacin (15 μg/ml) | 6.9 ± 0.4 | 7.9 ± 0.5 | 7.5 ± 0.3 |

TABLE 3
Changes in Expression of MAV-3013 and MAV 0831 (Antibiotic
added concomitant to bacteria at t = 0 h )

| | Biofilm (%) | Microaggregate (%) |
|---|---|---|
| No antibiotic | 100 ± 2 | 100 ± 3 |
| CFI (300 μg/ml) | 30 ± 7 | 38 ± 12 |
| Ciprofloxacin (300 μg/ml) | 91 ± 4 | 95 ± 6 |

Conclusion: CFI treatment delivered at the time of infection at concentrations that may be achievable in the respiratory tract in humans can inhibit gene expression leading to M. avium microaggregate formation and prevent biofilm formation.

### Example 4

### Time Dependency of Treatment Administration on M. avium Biofilm/Microaggregate Formation (Bermudez et al., 2016)

Introduction: A related study to Example 3 looked at the time-dependency of CFI and free ciprofloxacin administration on their ability to inhibit the formation of both biofilms and microaggregates of M. avium.

Methods: M. avium strains 104 and A5 (clinical isolates) were allowed to form biofilms and microaggregates on both plastic surfaces and the surface of a monolayer of HEp-2 cells, which are oropharyngeal epithelial cells, cultured in presence of RPMI-1640 medium. CFI or free ciprofloxacin (15 or 300 μg/mL ciprofloxacin) was added concomitant to bacteria at t=0 h, or 1 h, 2 h, 4 h, 12 h, 24 h, or 48 h following infection and then the CFU were measured. As mentioned above, both the 15 and 300 μg/mL concentrations are clinically relevant and achievable in sputum.

Results: For the biofilm on plastic model, while 15 μg/ml CFI significantly inhibited and reduced microaggregate formation (31%, 0<0.05) when added simultaneously (t=0 h) to the infection model versus untreated control, the 300 μg/ml CFI concentration significantly inhibited and reduced microaggregate formation (p<0.05) both when added at the 1-h post infection time point versus control (53%) and versus free ciprofloxacin (45%) as well as when added simultaneously (t=0 h) versus control (84%) and versus free ciprofloxacin (78%). There were no significant decreases in CFU with free ciprofloxacin at either concentration.

TABLE 4
Mean Efficacy against M. avium Strains 104 and A5
in an In Vitro Biofilm Model on Plastic Surface at
Ciprofloxacin Concentrations of 15 and 300 μg/mL

| | | | |
|---|---|---|---|
| Time | Control | CFI | Ciprofloxacin |
| (h) | (CFU) | (CFU) | (CFU) |

15 μg/ml

| | | | |
|---|---|---|---|
| 0 | 1 × 10⁶ | 6.9 ± 0.4 × 10⁵ * | 8.5 ± 0.3 × 10⁵ |
| 1 | 1 ± 0.2 × 10⁶ | 9.1 ± 0.3 × 10⁵ | 1.2 ± 0.3 × 10⁶ |
| 2 | 1.3 ± 0.6 × 10⁶ | 1.1 ± 0.5 × 10⁶ | 1.4 ± 0.5 × 10⁶ |
| 4 | 1.4 ± 0.5 × 10⁶ | 1.4 ± 0.3 × 10⁶ | 1.6 ± 0.3 × 10⁶ |
| 12 | 2.3 ± 0.4 × 10⁶ | 2.4 ± 0.5 × 10⁶ | 2.8 ± 0.4 × 10⁶ |
| 24 | 4.1 ± 0.2 × 10⁶ | 4.4 ± 0.3 × 10⁶ | 4.9 ± 0.3 × 10⁶ |

300 μg/ml

| | | | |
|---|---|---|---|
| 0 | 1 × 10⁶ | 1.6 ± 0.3 × 10⁵ *† | 7.3 ± 0.5 × 10⁵ |
| 1 | 1 ± 0.2 × 10⁶ | 4.7 ± 0.4 × 10⁵ *† | 8.6 ± 0.4 × 10⁵ |
| 2 | 1.3 ± 0.6 × 10⁶ | 7.9 ± 0.3 × 10⁵ | 1.5 ± 0.3 × 10⁶ |
| 4 | 1.4 ± 0.5 × 10⁶ | 1.5 ± 0.4 × 10⁶ | 1.7 ± 0.5 × 10⁶ |
| 12 | 2.3 ± 0.4 × 10⁶ | 2.5 ± 0.5 × 10⁶ | 2.5 ± 0.3 × 10⁶ |
| 24 | 4.1 ± 0.2 × 10⁶ | 4.7 ± 0.3 × 10⁶ | 4.1 ± 0.4 × 10⁶ |

* p < 0.05 vs. Control
* p < 0.05 vs. Control
† p < 0.05 vs. Free Ciprofloxacin

For the biofilm on epithelial cell monolayer model, which tested only the 300 μg/mL concentration, CFI significantly inhibited and reduced (p<0.05) CFU when added simultaneously versus control (99%) and versus free ciprofloxacin (79%), and up to 4 h post infection following infection versus control (71%) and versus free ciprofloxacin (75%)Error! Reference source not found. There were no significant decreases in CFU with free ciprofloxacin.

TABLE 5
Mean Efficacy against M. avium Strains 104 and A5
in an In Vitro Biofilm Model on Epithelial Cell Monolayer
at Ciprofloxacin Concentration of 300 μg/mL

| 300 μg/ml | | | |
|---|---|---|---|
| Time | Control | CFI | Ciprofloxacin |
| (h) | (CFU) | (CFU) | (CFU) |
| 0 | 6.9 ± 0.3 × 10⁶ | 8.1 ± 0.5 × 10⁴ *† | 3.8 ± 0.3 × 10⁵ |
| 1 | 7.3 ± 0.4 × 10⁵ | 1.7 + 0.4 × 10⁵ *† | 5.9 ± 0.5 × 10⁵ |
| 2 | 9.6 ± 0.3 × 10⁵ | 5.5 + 0.5 × 10⁵ *† | 9.1 ± 0.4 × 10⁵ |
| 4 | 2.1 ± 0.3 × 10⁶ | 6.1 ± 0.3 × 10⁵ *† | 2.4 ± 0.6 × 10⁶ |
| 12 | 3.9 ± 0.3 × 10⁶ | 3.9 ± 0.6 × 10⁶ | 3.8 ± 0.4 × 10⁶ |
| 24 | 4.5 ± 0.6 × 10⁶ | 4.6 ± 0.3 × 10⁶ | 4.8 ± 0.5 × 10⁶ |

* p < 0.05 vs. Control
† p < 0.05 vs. Ciprofloxacin

Conclusions: CFI treatment at a clinically relevant concentration (300 μg/mL) that is added concomitant to the bacterial infection at t=0 h and up to 1 h after infection for biofilm on plastic surface model and up to 4 h after infection for the epithelial cell monolayer model significantly inhibits M. avium microaggregate formation.

### Example 5

### Activity of Liposomal Ciprofloxacin Against M. avium Over 3 Weeks in M. avium Mouse Infection Model (Bermudez et al., 2015)

Rationale: Individuals with chronic lung pathology such as bronchiectasis, emphysema and cystic fibrosis frequently develop pulmonary infection caused by M. avium. The infection is characterized in the majority of the patients as peri-bronchiolar, with the development of granulomas. Treatment with the current recommended antibiotics is often insufficient to cure the condition. The efficacy of liposome-ciprofloxacin delivered by the respiratory route was evaluated.

Methods: CS7BL/6 mice (n=10/group) were infected by intranasal instillation (IN) with 5×10⁸ MAC 104 strain of M. avium. One week later, infection therapy via IN was initiated with CFI, Pulmaquin, or free ciprofloxacin at doses of 0.33, 0.67 and 1 mg/kg, which are clinically relevant in patients, or saline or empty liposome controls with the lipid dose matching that of the 1 mg/kg dose. Mice received therapy for 3 weeks, then were harvested and lungs and spleens were plated for bacterial counts.

Results: Over 3 weeks, there were no significant effects of empty liposomes and for free ciprofloxacin at 1 mg/kg had only a 19% decrease (p<0.05 versus saline control at 3 weeks) in the growth of M. avium in the lungs. In comparison, 1 mg/kg of Pulmaquin or CFI had larger significant reductions in lung CFU of 77% and 79%, respectively (p<0.05 for both versus saline control). These formulations also had significant decreases at 0.33 and 0.67 mg/kg. For Pulmaquin, these were 37% and 67%, respectively; for CFI, these were 45% and 57%, respectively, (p<0.05 for all versus saline control). However, there were no significant decreases for free ciprofloxacin at these doses. There were also no significant decreases in CFU in the spleen with any formulations or doses.

TABLE 6
Activity of Ciprofloxacin and Ciprofloxacin-liposome Preparations
against M. avium (MAC Strain 104) in Mice

| Experimental | | | | CFU | |
|---|---|---|---|---|---|
| Group | Time | Dose | | Lung | Spleen |
| Baseline | | | | 2.0 ± 0.4 × 10⁶ | 5.39 ± 0.4 × 10⁴ |
| Saline Control | 3 weeks | 0 | mg/kg | 1.06 ± 0.5 × 10⁷ | 4.72 ± 0.3 × 10⁵ |
| Empty Liposomes | 3 weeks | 0 | mg/kg ^{a} | 2.51 ± 0.4 × 10⁷ | 6.27 ± 0.4 × 10⁵ |
| Control | | | | | |
| Free ciprofloxacin | 3 weeks | 1 | mg/kg | 8.65 ± 0.4 × 10⁶ * | 6.47 ± 0.4 × 10⁵ |
| | | 0.67 | mg/kg | 1.04 ± 0.4 × 10⁷ | 5.99 ± 0.6 × 10⁵ |
| | | 0.33 | mg/kg | 2.64 ± 0.4 × 10⁷ | 7.38 ± 0.3 × 10⁵ |
| CFI | 3 weeks | 1 | mg/kg | 2.25 ± 0.4 × 10⁶ *^{,}** | 3.13 ± 0.4 × 10⁵ |
| | | 0.67 | mg/kg | 3.72 ± 0.5 × 10⁶ * | 7.25 ± 0.3 × 10⁵ |
| | | 0.33 | mg/kg | 5.84 ± 0.3 × 10⁶ * | 8.28 ± 0.4 × 10⁵ |
| Pulmaquin | 3 weeks | 1 | mg/kg | 2.47 ± 0.6 × 10⁶ *^{,}** | 6.14 ± 0.4 × 10⁵ |
| | | 0.67 | mg/kg | 3.49 ± 0.4 × 10⁶ * | 7.02 ± 0.3 × 10⁵ |
| | | 0.33 | mg/kg | 6.71 ± 0.3 × 10⁶ * | 8.30 ± 0.5 × 10⁵ |

* p < 0.05 versus saline and empty liposome controls
** p < 0.05 versus free ciprofloxacin
^{a} Dose of lipid for empty liposome control equals lipid dose of 1 mg/kg CFI

### Example 6

### Activity of Liposomal Formulations of Ciprofloxacin in the Lung Over 6 Weeks in M. avium Mouse Infection Model. (Bermudez et al., 2015)

Rationale: Individuals with chronic lung pathology such as bronchiectasis, emphysema and cystic fibrosis frequently develop pulmonary infection caused by M. avium. The infection is characterized in the majority of the patients as peri-bronchiolar, with the development of granulomas. Treatment with the current recommended antibiotics is often insufficient to cure the condition. The efficacy of liposome-ciprofloxacin delivered by the respiratory route was evaluated over a longer period (i.e., 6 weeks), since the treatment in humans is typically for many months.

Methods: C57BL/6 mice (n=10/group) were infected by IN with 1×10⁷ MAC 104 strain of M. avium. One week later, the mice received daily administration of therapies CFI, Pulmaquin and free ciprofloxacin at a dose 1 mg/kg or saline or empty liposome controls with the lipid dose matching that of the 1 mg/kg dose. Mice were harvested at weeks 3 and 6. Lungs were homogenized and plated to quantify the bacterial load.

Antimicrobial susceptibility: To verify the susceptibility of M. avium to ciprofloxacin, MAC 104 obtained before treatment and after treatment with CFI and free ciprofloxacin, their MICs were evaluated using a microdilution method.

Results: Table 7 shows the colonization of MAC 104 Strain of M. avium for each arm. Extending treatment of the 1 mg/kg dose for 6 weeks significantly reduced the CFU compared to 3 weeks. Specifically, compared to the CFU for the saline control at week 1, treatment with Pulmaquin significantly reduced CFU at 3 weeks by 45%, (p<0.05) and further by 70%, (p<0.05 vs. both saline and CFU at 3 weeks). Similarly, treatment with CFI significantly reduced CFU at 3 weeks by 49%, (p<0.05) and further by 78% at 6 weeks, (p<0.05 vs. saline and CFU at 3 weeks). However, free ciprofloxacin alone, as well as empty liposomes, did not have a statistically significant effect. Therefore, treatment with CFI and mixtures of free and encapsulated ciprofloxacin (Pulmaquin) were found to provide a statistically significant decrease in CFU in this mouse infection model, while free ciprofloxacin alone, as well as empty liposomes, did not have a statistically significant effect.

Results: The antimicrobial susceptibility of M. avium was unchanged with an MIC of 8 μg/mL before and after treatment with either Ciprofloxacin or CFI. Thus, treatment with CFI is not associated with resistance after 6 weeks of therapy. (Although Pulmaquin was not tested for resistance, similar results would be expected.

TABLE 7
Activity of Ciprofloxacin and Ciprofloxacin-liposome Formulations
against M. avium (MAC Strain 104) in Mice over 6 Weeks

| | | | | CFU, Lungs | |
|---|---|---|---|---|---|
| Experimental Group | Dose | | Baseline | 3 Weeks | 6 Weeks |
| Saline Control(1 week) | 0 | mg/kg | 1.7 ± 0.3 × 10⁶ | — | — |
| Empty Liposome | 0 | mg/kg ^{a} | — | 9.8 ± 0.4 × 10⁶ | 1.4 ± 0.5 × 10⁷ |
| Control | | | | | |
| Ciprofloxacin | 1 | mg/kg | — | 9.1 ± 0.5 × 10⁶ | 1.0 ± 0.3 × 10⁷ |
| CFI | 1 | mg/kg | — | 8.6 ± 0.4 × 10⁵ * | 3.8 ± 0.4 × 10⁵ *^{,}** |
| Pulmaquin | 1 | mg/kg | — | 9.4 ± 0.5 × 10⁵ * | 5.1 ± 0.3 × 10⁵ *^{,}** |

* p < 0.05 versus no treatment (baseline) and empty liposome control.
** p < 0.05 for 6 weeks versus 3 weeks
^{a} Dose of lipid for empty liposome control equals lipid dose for CFI

Conclusion: Liposome-Ciprofloxacin formulations, CFI and Pulmaquin are effective against M. avium infection in the lung in a mouse model of lung; CFI and liposomes containing nanocrystalline ciprofloxacin are also effective in a model of macrophage infection. These formulations are also superior to free ciprofloxacin and empty liposome controls. The efficacy of CFI and Pulmaquin improved with the duration of treatment with the decrease at Week 6 being significantly greater than that at Week 3; thus, there is progressive improvement with time. Since the treatment in humans is typically for many months these findings are encouraging. There was also no evidence of resistance or adverse findings.

### Example 7

### Activity of Liposomal Ciprofloxacin Against M. abscessus in Human Macrophage Model (Blanchard et al, 2014)

Rationale: Lung infections from M. abscessus rank second in incidence to those from M. avium and cause lung infections that are more severe making them more difficult and costly to manage (Ballarino et al, 2009; Prevots et al, 2010). Current therapy often fails or is associated with significant side effects (Griffith and Aksamit, 2012). In a recent Phase 2 clinical trial of liposomal amikacin for inhalation (ARIKAYCE™) in patients with treatment for refractory NTM infection, encouraging sputum conversion results were seen for M. avium but not for M. abscessus (Olivier et al, 2016; Winthrop et al, 2015).

Methods: The efficacy of liposomal ciprofloxacin was tested with an infected human macrophage model using monolayers of THP-1 human macrophages as in Example 2. The cells were infected with M. abscessus strain 101 or 102 (clinical isolates) by exposing them to bacteria for 1 h and then allowing the bacteria to replicate intracellularly for 18 h. Treatment was daily for 4 days and consisted of either CFI, or free ciprofloxacin at 10 and 20 μg/mL, which are concentrations encountered in treated lungs in humans, or buffer or empty liposome control with the concentration of lipids matching the concentration of lipids in the CFI (20 μg/mL). The number of viable intracellular bacteria (CFU) was then quantified on Day 4

Results: The results are shown in Table 8. For M. abscessus 101, CFI at 10 and 20 μg/mL significantly decreased CFU by ˜2 log, i.e., 98.4 and 99.1%, respectively (p<0.05 for both); whereas, the same concentrations of free ciprofloxacin had increases in CFU versus buffer control on Day 0. For M. abscessus 102, CFI at 10 and 20 μg/mL had essentially the same results, significantly decreasing CFU by ˜2 log, i.e., 98.4 and 99.0%, respectively (p<0.05 for both); whereas, the same concentrations of free ciprofloxacin again had increases in CFU versus buffer control on Day 0.

TABLE 8
Activity of CFI and Ciprofloxacin at 20 μg/mL against M. abscessus in Macrophages

| | | Concentration | CFU | |
|---|---|---|---|---|
| Bacterial strain | Treatment | (μg/mL) | Day 0 | Day 4 |
| M. abscessus 101 | Buffer Control | 0 | 3.2 ± 0.4 × 10⁵ | 3.6 ± 0.3 × 10⁶ |
| | Empty Liposome | 0 | | 3.9 ± 0.5 × 10⁶ |
| | Control^{a} | | | |
| | Ciprofloxacin | 10 | | 3.2 ± 0.4 × 10⁶ |
| | Ciprofloxacin | 20 | | 1.9 ± 0.5 × 10⁶ |
| | CFI | 10 | | 5.1 ± 0.4 × 10³ * |
| | CFI | 20 | | 3.0 ± 0.3 × 10³ * |
| M. abscessus 102 | None | | 4.1 ± 0.6 × 10⁵ | 3.3 ± 0.5 × 10⁶ |
| | Empty Liposome | 0 | | 4.6 ± 0.5 × 10⁶ |
| | Control^{a} | | | |
| | Ciprofloxacin | 10 | | 3.0 ± 0.5 × 10⁶ |
| | Ciprofloxacin | 20 | | 1.2 ± 0.3 × 10⁶ |
| | CFI | 10 | | 6.7 ± 0.3 × 10³ * |
| | CFI | 20 | | 4.2 ± 0.6 × 10³ * |

* p < 0.05 compared to the initial infecting load in macrophages on Day 0
^{a}Empty liposome control with the concentration of lipids matching the concentration of lipids in the CFI (20 μg/mL).

### Example 9

### Activity of Liposomal Ciprofloxacin Against Formation of M. abscessus Biofilms (Blanchard et al., 2014)

Rationale: M. abscessus forms biofilms; studies have demonstrated that the ability to form biofilm is associated with the efficiency of infection. It was investigated whether CFI was active against bacteria in biofilms formed from M. abscessus 105.

Method: Biofilms were allowed to establish for 24 days then treated for 72 hours with either CFI at 50 or 100 μg/mL or free ciprofloxacin at 100 μg/mL, which are all clinically relevant concentrations, or controls, which were buffer or empty liposome control with the concentration of lipids matching the concentration of lipids in the 100 μg/ml CFI. The biofilms were allowed to grow for another 24 hours and then the number of viable intracellular bacteria (CFU) were quantified (Day 4).

Results: The results for are shown in Table 9. CFI had decreases in CFU of 5% and 58%, respectively, which were significant for 100 μg/mL (p<0.05 versus buffer control on Day 0); whereas free ciprofloxacin at 100 μg/mL had only a 21% decrease (not significant).

TABLE 9
Effect of Treatment on M. abscessus 105 Biofilm

| | | Concentration | CFU | |
|---|---|---|---|---|
| Bacterial strain | Treatment | (μg/ml.) | Day 0 | Day 4 |
| M. abscessus 105 | Buffer Control | 0 | 3.8 ± 0.6 × 10⁷ | 3.9 ± 0.8 × 10⁷ |
| | Empty Liposome | 0 ^{a} | | 3.9 ± 0.6 × 10⁷ |
| | Control | | | |
| | Ciprofloxacin | 100 | | 3.0 ± 0.5 × 10⁷ |
| | CFI | 50 | | 3.6 ± 0.4 × 10⁷ |
| | CFI | 100 | | 1.6 ± 0.5 × 10⁷ * |

* p < 0.05 compared to the initial infecting load in macrophages
^{a} Concentration of lipids in empty liposomes match the concentration of lipids in the 200 μg/ml liposomal ciprofloxacin

### Example 10

### Activity of Liposomal Ciprofloxacin Against M. abscessus in Mouse Lung Infection Model (Blanchard et al, 2015)

Rationale: Lung infections from M. abscessus rank second in incidence to those from M. avium and cause lung infections that are more severe making them more difficult and costly to manage (Ballarino et al, 2009; Prevots et al, 2010). Current therapy often fails or is associated with significant side effects (Griffith and Aksamit, 2012). In a recent Phase 2 clinical trial of liposomal amikacin for inhalation (ARIKAYCE™) in patients with treatment for refractory NTM infection, encouraging sputum conversion results were seen for M. avium but not for M. abscessus (Olivier et al, 2016; Winthrop et al, 2015). The efficacy of liposome-ciprofloxacin delivered by the respiratory route was evaluated. The efficacy of liposome-ciprofloxacin delivered by the respiratory route in infected mice was evaluated.

Methods: C57 beige bg/bg mice (n=12/group per time point) were infected by IN with 5.4±0.3×107 M. abscessus 101. One week later (Week 0), therapy was initiated via IN with Pulmaquin, CFI, or free ciprofloxacin at a ciprofloxacin dose of 1 mg/kg, which is a clinically relevant dose, delivered daily for 3 and 6 weeks, the controls were saline and empty liposomes with the lipid dose matching the lipid content of the 1 mg/kg CFI dose. At the end of dosing, mice were harvested and lungs and spleens were plated for bacterial counts.

Results: The results are shown in Table 10. Compared to CFU for the saline control at week 0, treatment with Pulmaquin significantly reduced CFU in lungs at 3 weeks by 96.1%, (p<0.05) and further by 99.4% (>2 log), (p<0.05 vs. both saline and CFU at 3 weeks). Similarly, treatment with CFI significantly reduced CFU in lungs at 3 weeks by 95.2%, (p<0.05) and further at 6 weeks by 99.7% (˜3 log), (p<0.05 vs. saline and CFU at 3 weeks). The decreases with free ciprofloxacin were smaller (2% and 26% at 3 and 6 weeks, respectively), and not statistically significant. There were also significant effects in the spleen (data not shown).

TABLE 10
Efficacy of Ciprofloxacin and Ciprofloxacin-liposome Preparations
against M. abscessus 101 in Mice over 3 and 6 Weeks

| | | | | CFU, Lungs | |
|---|---|---|---|---|---|
| Experimental Group | Dose | | Baseline | 3 Weeks | 6 Weeks |
| Saline Control(1 week) | 0 | mg/kg | 2.6 ± 0.4 × 10⁶ | — | 5.4 ± 0.6 × 10⁵- |
| Empty Liposome | 0 | mg/kg ^{a} | — | 4.8 ± 0.5 × 10⁵ | 3.6 ± 0.3 × 10⁵ |
| Control | | | | | |
| Ciprofloxacin | 1 | mg/kg | — | 5.3 ± 0.3 × 10⁵ | 3.6 ± 0.3 × 10⁵ |
| CFI | 1 | mg/kg | — | 2.6 ± 0.6 × 10⁴ * | 1.4 ± 0.5 × 10³ *^{,}** |
| Pulmaquin | 1 | mg/kg | — | 2.1 ± 0.4 × 10⁴ * | 3.0 ± 0.4 × 10³ *^{,}** |

* p < 0.05 versus no treatment (baseline) and empty liposome control.
** p < 0.05 for 6 weeks versus 3 weeks
^{a} Dose of lipid for empty liposome control equals lipid dose for CFI

Conclusions: Both 3- and 6-week treatment with Pulmaquin and CFI at clinically relevant doses using mice with lung infections from M. abscessus resulted in significant reductions of bacterial load in the lungs, with the decrease at Week 6 being significantly greater than that at Week 3; thus, there is progressive improvement with time. There was also no evidence of resistance or adverse findings. Since the treatment in humans is typically for many months these findings are encouraging.

### REFERENCES

Each of the following is incorporated by reference.
Adjemian J, Olivier K N, Seitz A E, Holland S M, Prevots D R, Prevalence of nontuberculosis mycobacterial lung disease in US Medicare beneficiaries. Am J Respir Crit Care Med, 185: 881-886, 2012. PMID: 22773732
Ballarino G J, Olivier K N, Claypool R J, Holland S M, Prevots D R. Pulmonary nontuberculous mycobacterial infections: antibiotic treatment and associated costs. Respir Med. 2009; 10):1448-1455.
Bangham A D, Standish M M, Watkins J C, Diffusion of univalent ions across the lamellae of swollen phospholipids. J Mol Biol. 13 (1) (1965) 238-252.
Barker A F, Couch L, Feil S B, Gotfried M H, Ilowite J, Meyer K C, O'Donnell A, Sahn S A, Smith L J, Stewart J O, Abuan T, Tully H, Van Dalfsen J, Wells C D, Quan J. Tobramycin solution for inhalation reduces sputum Pseudomonas aeruginosa density in bronchiectasis. Am J Respir Crit Care Med 162: 481-5, 2000. PMID: 10934074
Bergogne-Bérézin E. Pharmacokinetics of fluoroquinolones in respiratory tissues and fluids. Quinolones Bull. 10: 1-18, 1993.
Bermudez L E, Motamedi N, Kolonoski P. Chee C, Baimukanova G, Bildfell R, Wang G, Phan L T, Young LS. The efficacy of clarithromycin and the bicyclolide EDP-420 against Mycobacterium avium in a mouse model of pulmonary infection. J Infect Dis, 197: 1506-10, 2008. PMID: 18422455
Bermudez L E, Blanchard J D, Hauck L, Gonda I. Treatment of Mycobacterium avium subsp hominissuis (M. avium) lung infection with liposome-encapsulated ciprofloxacin resulted in significant decrease in bacterial load in the lung. Presented at American Thoracic Society (ATS) International Conference, Denver, Colo. 2015. Am J Respir Crit Care Med. 2015; 191: A6293
Bermudez L E, Blanchard J, Babrak L, Gonda I. Liposome-ciprofloxacin inhibits Mycobacterium avium subs hominissuis (M. avium) microaggregate formation in a dose and time dependent manner. Presented at American Thoracic Society (ATS) International Conference, San Francisco, Calif. 2016. Am. J. Respir. Crit. Care Med. 2016; 193; A3734
Bilton D, P Bruinenberg P, Otulana B. Inhaled liposomal ciprofloxacin hydrochloride significantly reduces sputum Pseudomonas aeruginosa density in CF and non-CF bronchiectasis. Presented at American Thoracic Society (ATS) International Conference, San Diego, Calif. Am J Respir Crit Care Med 179:A3214, 2009.
Bilton D, Bruinenberg P, Otulana B, Morishige R, Blanchard J, DeSoyza A, Serisier D. Inhaled liposomal ciprofloxacin hydrochloride significantly reduces sputum Pseudomonas aeruginosa density in CF and non-CF bronchiectasis. Presented at European Respiratory Society (ERS) Conference. Abstract 1362, 2009.
Bilton D, De Soyza A, Hayward C, Bruinenberg P. Effect of a 28-day course of two different doses of once a day liposomal ciprofloxacin for inhalation on sputum Pseudomonas aeruginosa density in non-CF bronchiectasis, Presented at American Thoracic Society (ATS) International Conference, New Orleans, La. Am J Respir Crit Care Med 181:A3191, 2010.

Bilton D, Serisier D J, DeSoyza A, Wolf R, Bruinenberg P. Multicenter, randomized, double-blind, placebo-controlled study (ORBIT 1) to evaluate the efficacy, safety, and tolerability of once daily ciprofloxacin for inhalation in the management of Pseudomonas aeruginosa infections in patients with non-cystic fibrosis bronchiectasis. Presented at European Respiratory Society Annual Congress, Amsterdam, The Netherlands. Abstract 1925, 2011.
Blanchard J D. Pulmonary drug delivery as a first response to bioterrorism. In: Dalby R N, Byron P R, Peart J, Suman J D, and Farr S J, eds., Respiratory Drug Delivery X, River Grove, Ill.: Davis Healthcare International, 2006, 73-82.
Blanchard J, Danelishvili L, Gonda I, Bermudez L. Liposomal ciprofloxacin preparation is active against Mycobacterium avium subsp hominissuis and Mycobacterium abscessus in macrophages and in biofilm. Presented at American Thoracic Society (ATS) International Conference, San Diego, Calif. 2014. Am. J. Respir. Crit. Care Med. 2014; 189: A6677.
Blanchard J, Elias V, Gonda I, Bermudez LE. Treatment of lung infection caused by Mycobacterium abscessus in beige mice with pulmonary delivery of liposomally encapsulated ciprofloxacin is associated with significant reduction of bacterial load. Presented at Interscience Conference on Antimicrobial Agents and Chemotherapy (ICAAC), San Diego, Calif., 2015. Poster B-536.
Bruinenberg P, Otulana B, Blanchard J, Morishige R, Cipolla D, Wilson J, Serisier D. The effect of once-a-day, inhaled liposomal ciprofloxacin hydrochloride for inhalation on bacterial density in cystic fibrosis patients with chronic P. aeruginosa infection. Presented at North American Cystic Fibrosis Conference, Orlando, Fla., 2008. Ped Pulmon 43 (Suppl 31):401, 2008.
Bruinenberg P, Otulana B, Blanchard J, Cipolla D, Wilson J, Serisier D. Pharmacokinetics and antibacterial activity of inhaled liposomal ciprofloxacin hydrochloride in healthy volunteers and in cystic fibrosis (CF) patients. Presented at 32nd European Cystic Fibrosis Conference, Brest, France. J Cystic Fibrosis 8 (Suppl 2):549, 2009.
Bruinenberg P, Blanchard J, Cipolla D, Serisier D. Safety, tolerability and pharmacokinetics of novel liposomal ciprofloxacin formulations for inhalation in healthy volunteers and in non-cystic bronchiectasis patients. Presented at American Thoracic Society (ATS) International Conference, New Orleans, La. Am J Respir Crit Care Med 181:A3192, 2010.
Bruinenberg, P, Blanchard J D, Cipolla D C, Dayton F, Mudumba S, Gonda I. Inhaled liposomal ciprofloxacin: once a day management of respiratory infections. In: Dalby, R N, Byron P R, Peart J, Suman J D, Farr S J, Young P M, eds. Respiratory Drug Delivery 2010. River Grove, Ill.: Davis Healthcare International, 73-81, 2010.
Bruinenberg P, Serisier D, Blanchard J, Cipolla D, Gonda I. Effects and modulation of release rate of inhaled ciprofloxacin with liposomal formulations in healthy subjects and patients with bronchiectasis. Presented at European Respiratory Society Annual Congress, Barcelona, Spain. Abstract 1625, 2010.
Bruinenberg P, Serisier D, Cipolla D, Blanchard J. Safety, tolerability pharmacokinetics and antimicrobial activity of inhaled liposomal ciprofloxacin formulations in humans. Presented at North American Cystic Fibrosis Conference, Baltimore, Md. 45 (Suppl 33): Poster 377, 2010.
Bruinenberg P, Serisier D, Cipolla D, Blanchard J. Safety, tolerability, and pharmacokinetics of novel liposomal ciprofloxacin formulations in healthy volunteers (HV) and non-cystic fibrosis bronchiectasis (BE) patients. J Cystic Fibrosis 10 (Suppl 1): S29, 2011.
Carter G, Drummond D, Bermudez L E. Characterization of biofilm formation by Mycobacterium avium strains. J Med Microbiol 52: 747-52, 2003. PMID: 12909649
Chiu J, Nussbaum J, Bozzette S, Tilles J G, Young L S, Leedom J, Heseltine P N, McCutchan J A. Treatment of disseminated Mycobacterium avium complex infection in AIDS with amikacin, ethambutol and ciprofloxacin. Ann Intern Med 113: 358-61, 1990. PMID: 2382918
Cipolla D C, Dayton F, Fulzele S, Gabatan E, Mudumba S, Yim D, Wu H and Zwolinski R. (2010), Inhaled Liposomal Ciprofloxacin: In Vitro Properties and Aerosol Performance. Respiratory Drug Delivery 2010. pp. 409-414. Editors, Richard N. Dalby, Peter R. Byron, Joanne Peart, Julie D. Suman, Stephen J. Farr, Paul M. Young. Davis Healthcare Int'l Publishing, River Grove, Ill. Orlando, Fla., Apr. 25-29, 2010.
Cipolla D, Redelmeier T, Eastman S., Bruinenberg P, and Gonda I. (2011) Liposomes, niosomes and proniosomes—a critical update of their (commercial) development as inhaled products. Respiratory Drug Delivery Europe 2011, pp 41-54. Editors, Richard N. Dalby, Peter R. Byron, Joanne Peart, Julie D. Suman, Stephen J. Farr, Paul M. Young. Davis Healthcare Int'l Publishing, River Grove, Ill. Berlin, Germany, May 3-6, 2011.
Cipolla D, Wu H, Chan J, Chan H-K, and Gonda I. (2013a) Liposomal Ciprofloxacin for Inhalation Retains Integrity Following Nebulization. Respiratory Drug Delivery Europe 2013, pp 237-242. Editors, Richard N. Dalby, Peter R. Byron, Joanne Peart, Julie D. Suman, Stephen J. Farr, Paul M. Young. Davis Healthcare Int'l Publishing, River Grove, Ill. Berlin, Germany, May 21-24, 2013.
Cipolla D, Gonda I, and Chan H-K. (2013b) Liposomal Formulations for Inhalation. Therapeutic Delivery. Vol. 4, No. 8, pp. 1047-1072. doi: 10.4155/tde.13.71.
Cipolla D, Blanchard J, Gonda I. Development of Liposomal Ciprofloxacin to Treat Lung Infections. Pharmaceutics. 2016. 8(1), 6. doi: 10.3390/pharmaceutics 8010006
Conley J, Yang H, Wilson T, Blasetti K, Di Ninno V, Schnell G, Wong J P. Aerosol delivery of liposome encapsulated ciprofloxacin: aerosol characterization and efficacy against Francisella tuleransis infection in mice. Antimicrob Agents Chemother 41: 1288-92, 1997. PMCID: PMC163901
Costerton J W, Lewandowski Z, Caldwell D E, Korber D R, Lappin-Scott H M., Microbial biofilms. Annu Rev Microbiol. 1995; 49:711-45.
de Lalla F, Maserati R, Scarpellini P, Marone P, Nicolin R, Caccamo F, Rigoli R. Clarithromycin-ciprofloxacin-amikacin for therapy of Mycobacterium avium-Mycobacterium intracellulare bacteremia in patients with AIDS. Antimicrob Agents Chemother 36: 1567-9, 1992. PMCID: PMC191622
Di Ninno V L, Cherwonogrodzky J W, Wong J P. Liposome-encapsulated ciprofloxacin is effective in the protection and treatment of Balb/c mice against Francisella tularensis. J Infect Dis 168:793-794, 1993. PMID: 8354928
Fiel S B. Aerosolized antibiotics in cystic fibrosis: current and future trends. Expert Rev Respir Med 2: 479-487, 2008. PMID: 20477211
Finlay W H and Wong J P. Regional lung deposition of nebulized liposome-encapsulated ciprofloxacin. Int J Pharm. 1998, 167:121-127.
Fitzsimmons S C. The changing epidemiology of cystic fibrosis. J Pediatr. 1993 January; 122(1):1-9.
Fowler S J, French J, Scranton N J, Foweraker J, Condliffe A, Haworth C S, Exley A R, Bilton D. Nontuberculous mycobacteria in bronchiectasis: prevalence and patient characteristics. Eur Respir J 28: 1204-10, 2006. PMID: 16807259
Gilbert P, Das J, Foley I., Biofilm susceptibility to antimicrobials. Adv Dent Res. 1997 April; 11(1):160-7.
Griffith D E and Aksamit T. Therapy of refractory nontuberculous mycobacterial lung disease. Curr Opin Infect Dis. 2012 April; 25(2):218-27.
Hamblin K A, Blanchard J D, Atkins H S. Efficacy of liposome-encapsulated ciprofloxacin against Francisella tularensis Schu S4 strain. Presented at Interscience Conference on Antimicrobial Agents and Chemotherapy (ICACC), Chicago, Ill., Poster F1-741, 2011.
Hamblin K A, Armstrong S J, Kay B, Barnes K B, Davies C, Wong J P, Blanchard J D, Harding S V, Simpson A J H, Atkins H S. Liposome-encapsulation of ciprofloxacin improves protection against highly resistant Francisella tularensis Schu S4 strain. Antimicrob Agents Chemother 2014 (In press) PMID: 24637682.
Henkle E, Hedberg K, Schafer S, Novosad S, Winthrop K L. Population-based incidence of pulmonary nontuberculous mycobacterial disease in Oregon 2007 to 2012. Ann Am Thorac Soc. 2015; 12:642-647.
Inderlied C, Kolonoski P, Wu M, Young L. Amikacin, ciprofloxacin, and imipenem treatment for disseminated Mycobacterium avium complex infection of beige mice. Antimicrob Agents Chemother 33: 176-80, 1989. PMCID: PMC171452
Inderlied C B, Kemper C A, Bermudez L E. The Mycobacterium avium complex. Clin Micro Rev 6: 266-310, 1993. PMCID: PMC358286
Islam M S, Richards J P, Ojha A K. Targeting drug tolerance in mycobacteria: a perspective from mycobacterial biofilms. Expert Rev Anti Infect Ther. 10(9): 1055-1066 (2012).
Jordao L, Bleck C K, Mayorga L, Griffths G, Anes E. On the killing of mycobacteria by macrophages. Cell Microbiol 10: 529-48, 2008. PMID: 17986264
Lode H, Borner K, Koeppe P, Schaberg T., Azithromycin—review of key chemical, pharmacokinetic and microbiological features. J Antimicrob Chemother. 1996; 37, Suppl C: 1-8
Majumdar S, Flasher D, Friend D S, Nassos P, Yajko D, Hadley W K, Düzgünş N. Efficacies of liposome-encapsulated streptomycin and ciprofloxacin against Mycobacterium avium-M. intracellulare complex infections in human peripheral blood monocytes/macrophages. Antimicrob Agents Chemother 36: 2808-15, 1992. PMCID: PMC245550.
McNabe M, Tennant R, Danelishvili L, Young L, Bermudez L E. Mycobacterium avium ssp. hominissuis biofilm is composed of distinct phenotypes and influenced by the presence of antimicrobials. Clin Microbiol Infect 17(5) 697-703 (2012). PMID: 20636426
Meers P, Neville M, Malinin V, Scotto A W, Sardaryan G, Kurumunda R, Mackinson C, James G, Fisher S, Perkins W R. Biofilm penetration, triggered release and in vivo activity of inhaled liposomal amikacin in chronic Pseudomanas aeruginosa lung infections, J Antimicrob Chemother, 61: 859-68, 2008. PMID: 18305202
Niven RN and Schreier H. Nebulization of Liposomes: I. Effects of Lipid Composition. Pharm Res. 1990; 7:1127-1133.
Oh Y K, Nix D E, Straubinger R M. Formulation and efficacy of liposome-encapsulated antibiotics for therapy of intracellular Mycobacterium avium infection. Antimicrob Agents Chemother 39: 2104-11, 1995. PMCID: PMC162889
Olivier K N, Gupta R, Daley C L et al. Randomized trial of Liposomal Amikacin for Inhalation in patients with nontuberculous mycobacterial lung disease. Am J Respir Crit Care Med 2017; 195: 814-823.
Polonio R E Mermel L A, Paquette G E, Sperry J F., Eradication of biofilm-forming Staphylococcus epidermidis (RP62A) by a combination of sodium salicylate and vancomycin. Antimicrob Agents Chemother. 2001 November; 45(11):3262-6.
Prevots R D, Shaw P A, Strickland D, Jackson L A, Raebel M A, Blosky M A, Montes de Oca R, Shea Y R, Seitz A E, Holland S M, Olivier, K N. Nontuberculosis mycobacterial lung disease prevalence at four integrated health care delivery systems. Am J Respir Crit Care Med 182: 970-976, 2010 PMID: 20538958.
Serisier D J, Bilton D, De Soyza A, Thompson P J, Kolbe J, Greville H W, Cipolla D, Bruinenberg P, and Gonda I. (2013) Inhaled, Dual-Release Liposomal Ciprofloxacin in Non-Cystic Fibrosis Bronchiectasis (ORBIT-2)—a Randomised, Double-Blind, Placebo-Controlled Trial. Thorax. Vol. 68, No. 9, pp. 812-817 doi: 10.1136/thoraxjnl-2013-203207.
Shafran S D, Singer J, Zarowny D P, Phillips P, Salit I, Walmsley S L, Fong I W, Gill M J, Rachlis A R, Lalonde R G, Fanning M M, Tsoukas C M. A comparison of two regimens for the treatment of Mycobacterium avium complex bacteremia in AIDS, rifampin, ethambutol, and clarithromycin, versus rifampin, ethambutol, clofazimine and ciprofloxacin. N Engl J Med 335: 377-83, 1996. PMID: 8676931
Smith A L, Ramsey B W, Hedges D L, Hack B, Williams-Warren J, Weber A, Gore E J, Redding G J. Safety of aerosol tobramycin administration for 3 months to patients with cystic fibrosis. Ped Pulmonol. 1989; 7(4):265-271.
Van Heeckeren A M, Schluchter M D., Murine models of chronic Pseudomonas aeruginosa lung infection. Lab Anim. 2002 July; 36(3):291-312.
Van Heeckeren A M, Tscheikuna J, Walenga R W, Konstan M W, Davis P B, Erokwu B, Haxhiu M A, Ferkol T W. Effect of Pseudomonas infection on weight loss, lung mechanics, and cytokines in mice. Am J Respir Crit Care Med. 2000 January; 161(1):271-9.
Weber A, Smith A, Williams-Warren J, Ramsey B, Covert D S., Nebulizer delivery of tobramycin to the lower respiratory tract. Pediatr Pulmonol. 1994 May; 17(5):331-9.
Winthrop K L, Eagle G, McGinnis J P et al. Subgroup analyses of baseline demographics and efficacy in patients with refractory nontuberculous mycobacteria (NTM) lung infection treated with Liposomal Amikacin for Inhalation (LAI). Am J Respir Crit Care Med 2015; 191: A6294
Wong J P, Yang H, Blasetti K L, Schnell G, Conley J, Schofield L N. Liposome delivery of ciprofloxacin against intracellular Francisella tularensis infection. J Control Release 92: 265-73, 2003. PMID: 14568408
Wong J P, Schnell G, Simpson M, Saravolac E. Effects of liposome-encapsulated ciprofloxacin on phagocytosis, nitric oxide, and intracellular killing of Staphylcoccus aureus by murine macrophages. Artific Cells Blood Substit Immobil Biotechnol 28: 415-28, 2000. PMID: 11009114
Wolter J, Seeney S, Bell S, Bowler S, Masel P, McCormack J., Effect of long term treatment with azithromycin on disease parameters in cystic fibrosis: a randomised trial. Thorax 2002; 57: 212-216.
Yamazaki Y, Danelishvili L, Wu M, Hidaka E, Katsuyama T, Stang B, Petrofsky M, Bildfell R, Bermudez L. The ability to form biofilm influences Mycobacterium avium invasion and translocation of bronchial epithelial cells. Cell Microbiol 8: 806-14, 2006. PMID: 16611229
Yeager H, Raleigh J W. Pulmonary disease due to Mycobacterium intracellulare. Am Rev Resp Dis 108: 547-52, 1973. PMID: 4745250

1. A method of inhibiting formation of microaggregates of nontuberculous mycobacteria (NTM), comprising:
administering to a patient a formulation comprising an antibiotic in unilamellar Liposomes.

2. The method of claim 1, wherein the liposomes are comprised of hydrogenated soy phosphatidylcholine (HSPC) and cholesterol.

3. The method of claim 1, wherein the liposomes are comprised of dipalmitoylphosphatidylcholine (DPPC) and cholesterol

4. The method of claim 1, wherein the liposomes have a mean diameter of 10 nanometers to 5.0 microns.

5. The method of claim 1, wherein the liposomes have a mean diameter of 50 to 300 nanometers.

6. The method of claim 1, wherein the antibiotic is ciprofloxacin.

7. The method of claim 1, comprising 20 to 80 mg/mL ciprofloxacin.

8. The method of claim 1, comprising 30 to 70 mg/mL ciprofloxacin.

9. The method of claim 1, comprising 40 to 60 mg/mL ciprofloxacin.

10. The method of claim 1, wherein the composition is aerosolized.

11. The method of claim 1, wherein particles of aerosolized have an aerodynamic diameter in the range of 0.5 to 12 microns.

12. The method of claim 1, wherein the aerosolized composition is inhaled into lungs of a human patient.

13. The method of claim 1, further comprising 40 to 100 mg/mL amikacin.

14. The method of claim 1, for use in inhibiting formation of microaggregates of nontuberculous mycobacteria (NTM) in biofilms.

15. A method of treatment, comprising:
identifying a patient as susceptible to the formation of non-tuberculosis microbacteria (NTM) microaggregate formation; and
administering to the patient a formulation comprising a pharmaceutically acceptable carrier, free unencapsulated ciprofloxacin, and liposomal ciprofloxacin comprising 20 to 80 mg/mL ciprofloxacin in unilamellar liposomes comprised of cholesterol and hydrogenated soy phosphatidylcholine (HSPC).
